(19) United States
(12) Patent Application Publication
(10) Pub. No.: US 2022/0133944 A1
(43) Pub. Date: May 5, 2022

(54) PASTE FOR MARKING TEXTILE FABRICS AND/OR OTHER PRODUCTS INCAPABLE OF X-RAY CONTRAST

(71) Applicant: Speed Care Mineral GmbH, Neubrandenburg (DE)

(72) Inventors: Joachim SCHOMBURG, Neubrandenburg (DE); Christian SCHULTZ, Neubrandenburg (DE); Wolfgang FROHN, Schauenstein (DE)

(73) Assignee: Speed Care Mineral GmbH, Neubrandenburg (DE)

(21) Appl. No.: 17/434,217

(22) PCT Filed: Feb. 3, 2020
(86) PCT No.: PCT/EP2020/052573

§ 371 Date: Aug. 26, 2021

(30) Foreign Application Priority Data
Feb. 28, 2019 (DE) 10 2019 105 111.8

(51) Int. Cl. A61L 15/54; A61L 15/18

(52) U.S. Cl. CPC A61L 15/54; A61L 15/18

(57) ABSTRACT

The invention relates to a paste for marking textile fabrics and/or other products incapable of x-ray contrast. The paste comprises the mineral materials barite (BaSO₄) and halloysite. These substances have a ratio of four volume parts barite to one volume part halloysite. The invention also relates to a wound dressing. The wound dressing contains composite fibers and is provided with the paste described at the outset. The invention additionally relates to the use of this wound dressing for the care of wound lesions. The invention also relates to a method for applying the paste described at the outset to a textile fabric and/or other products. The paste is applied to a textile fabric and/or to a different product by means of a printing process.

### TECHNICAL AREA

The invention relates to a paste for marking textile fabrics and/or other products incapable of x-ray contrast. The paste comprises the mineral materials barite (BaSO₄) and halloysite. Furthermore, the invention relates to a wound dressing and its use to care for wound lesions. The wound dressing contains composite fibers and is provided with the paste described at the outset. In addition, the invention relates to a method for applying the paste described at the outset to a textile fabric and/or other products.

### BACKGROUND AND PRIOR ART

A large number of wound dressings for wound lesions are known in the prior art. Many means for treating wound lesions comprise, for example, bandages made of cotton or other materials. Furthermore, there are so-called gauze bandages, which include a carrier gauze which is coated with chitosan. Furthermore, styptic bandages are known, which are coated using kaolin, which accelerates the blood coagulation. In addition to kaolin, zeolite granulates, three-layer silicates, or diatomaceous earth can be used for coating bandages. A further mineral-based wound dressing is known from WO 2019/016367 A1. The wound dressing disclosed there comprises composite fibers which are coated using halloysite, wherein the wound dressing has good properties for the styptic effect in the wound treatment.

However, such wound dressings have the disadvantage that they cannot be detected during x-ray examinations in medicine. X-ray diagnostics is a widespread imaging method, in which an x-ray is used for transmission through a body or parts of a body. The penetration of the body using x-rays is displayed in images which are referred to as x-ray images. In medicine, x-raying is used to establish anomalies in the body, which enable a diagnosis in conjunction with symptoms, signs, and possibly other examinations (x-ray diagnostics). The differing density of tissue of the human (or animal) body absorbs the x-rays at different strengths, so that an image of the body interior is achieved. To recognize wound dressings on an x-ray image in the case of intracorporeal applications, for example, the wound dressings also have to have properties capable of x-ray contrast.

The absorption of x-ray radiation is dependent on its energy level and increases with the number of the atoms “lying in the way”, i.e., the thickness of the object and its atomic density and the atomic number Z and mass number A of the atoms of the material. Contrast agents have a high level of absorption of the x-rays and are thus visible in the x-ray image. Barium sulfate (BaSO₄) and iodine compounds are contrast agents frequently used in medicine in particular.

Wound dressings capable of x-ray contrast are known from U.S. Pat. Nos. 4,718,897 A1, 4,185,626 A, EP 2567683 A1, EP 21 47046 B1, DE 19545289 A1, and U.S. Pat. No. 4,639,253 A. In the cited documents, thermoplastics comprising BaSO₄ are used, which are extruded as fibers, threads, or yarns and are heated at a temperature of 100° C. to 150° C., so that they are polymerized and are applied to plastics or textiles.

Thermoplastics, which are applied to textiles, are also described in documents DE 19857149 A1, DE 19940862 A1, and EP 1141129 A1. The disclosed thermoplastics comprise iodine compounds as contrast agents.

EP 0 272 901 B1 discloses a suspension capable of x-ray contrast, which is applied via printing methods to a wound dressing. The suspension contains a large fraction of thermoplastics, so that they are subsequently polymerized in a heating process.

The embodiments capable of x-ray contrast described in the prior art have the disadvantage of the high energy consumption due to the heating of the material to polymerize the thermoplastics.

Furthermore, thermoplastics have particularly negative aspects with respect to the environment. The production of thermoplastics requires a high consumption of nonrenewable resources such as petroleum or natural gas. Moreover, thermoplastics are not biologically degradable, so that they cause damage upon penetration into the environment. Thermoplastics can only be decomposed and pulverized, whereby they continue to exist as microplastics and can thus also enter the food chain.

A further disadvantage of the use of thermoplastics can result in that under certain circumstances they can result in a physical incompatibility. In the case of the intracorporeal application of a wound dressing, the thermoplastics can possibly induce inflammations or also be carcinogenic.

In addition to medicine, further areas of application of x-rays also exist. Thus, x-rays are used in the food industry, for example, to monitor packaged products for unintended contents. Furthermore, in the airline industry, baggage checks are carried out on the basis of x-rays. Means are not known in any of these areas of application to identify materials incapable of x-ray contrast so that they are visible in an x-ray image. These industries thus also have a demand for a marking which identifies objects incapable of x-ray contrast under x-rays. For example, in the airline industry, an object which is already assessed by security personnel and classified as harmless can be marked before the use of x-rays. Subsequently, it could be assessed in the x-ray image, in which this object would be classified as hazardous without marking, as already checked and assessed as harmless.

### OBJECT OF THE INVENTION

The object of the invention is thus to remedy the disadvantages of the prior art and provide an environmentally-protective and low-power-consumption means which ensures the detectability of textile fabrics and/or other products incapable of x-ray contrast in the x-ray image.

### SUMMARY OF THE INVENTION

The object according to the invention is achieved by the features of the independent claims. Advantageous embodiments of the invention are described in the dependent claims.

The invention relates in a first aspect to a paste for marking textile fabrics and/or other products incapable of x-ray contrast, characterized in that the paste includes mineral materials comprising barite (BaSO₄) and halloysite in a ratio of four volume parts barite to one volume part halloysite.

The paste is particularly advantageously capable of x-ray contrast and environmentally protective, since in contrast to comparable pastes of the above-described prior art it does not comprise thermoplastics. Surprisingly, it has been shown at precisely this ratio of barite to halloysite that the paste is very well visible in an x-ray image and nonetheless the advantageous properties of the halloysite in medical use are achieved. The mineral halloysite advantageously has a styptic effect. The use of the only relatively small quantities of the halloysite according to the invention in relation to the barite proves to be cost-effective, since the halloysite is a very rare mineral, which is difficult to acquire, in contrast to barite. The ratio according to the invention additionally enables surprisingly rapid and advantageously uniform thorough mixing of the halloysite with the barite. In addition to the mentioned advantages of this paste according to the invention, surprisingly few further components are needed for the later permanent fixation of the paste on the textile fabrics and/or other products incapable of x-ray contrast. These components will be explained in detail in the further course of the description.

It is apparent to a person skilled in the art here that the described volume components essentially mean four volume components barite to essentially one volume component halloysite. Accordingly, the ratios such as, for example 3.5; 3.6 . . . 3.99 . . . 4.1 . . . 4.999 volume components barite to 0.5; 0.6; . . . 1.999 volume components halloysite also fall under the described formulation.

Terms such as essentially, approximately, about, circa, etc. preferably describe a tolerance range of less than ±40%, preferably less than ±20%, particularly preferably less than ±10%, even more preferably less than ±5%, and in particular less than ±1% and always comprise the exact value. Similar describes preferred variables which are approximately equal. Partially preferably describes up to at least 5%, particularly preferably at least 10%, and in particular at least 20%, in some cases up to at least 40%.

In the meaning of the invention, a paste is preferably a salve, a cream, a gel, and/or a solid-liquid mixture (suspension) having a high content of solids. The proportion of solids in relation to a total volume or a total weight is preferably ≥15%. The viscosity of the paste at 20° C. and at ambient pressure is preferably between 5 mPa s and 2×10⁶ mPa s, more preferably between 200 mPa s and 3000 mPa s, and particularly preferably between 500 mPa s and 1500 mPa s, and in particular between 550 mPa s and 650 mPa s. The advantage of a paste or its good properties with respect to its flowing capability and further processing, so that the paste according to the invention is particularly well suitable for marking textile fabrics and/or other products incapable of x-ray contrast. Moreover, the production of such a paste is possible without great expense (inter alia, power consumption). In addition, all paste properties can be varied or adapted depending on the area of use with small changes in the composition of the paste.

In one preferred embodiment, textile fabrics are selected from a group comprising cables, woven fabrics, meshes, knitted fabrics, netting, stitch-bonded fabrics, nonwoven materials, and felts. Such textile fabrics are known to a person skilled in the art. Textile fabrics are flexible products which represent many possible uses and are particularly well suitable for use as a wound dressing. Due to their flexible property, they may particularly advantageously be applied flexibly to all body parts of humans and animals. Moreover, wound dressings ensure an advantageous healing-promoting moist wound climate and are capable of absorbing many wound liquids such as blood or the like.

In the meaning of the invention, products incapable of x-ray contrast are those materials which are not visible under the effect of x-ray radiation in an x-ray image resulting therefrom. The products can preferably be made, for example, of paper, wood, plastic, etc.

Barite or (BaSO₄) is also known under the name barium sulfate. Barite is known to a person skilled in the art and has advantageous properties with respect to the absorption of x-rays. In addition, barite is a good filler. It increases the surface hardness of plastics. Moreover, barite is a frequently occurring mineral, so that it also introduces its advantageous properties under cost-effective aspects into the paste according to the invention.

Halloysite is associated with the mineral class of silicates. Within the silicate minerals, halloysite is a representative of the layered silicates (phyllosilicates) and moreover is part of the group of clay minerals. Halloysite is the eponymous mineral of the halloysite group, which consists of the minerals halloysite-7 Å, halloysite-10 Å, and hisingerite. Halloysite-10 Å is also known to a person skilled in the art under the name endellite. Halloysites are bipolar, i.e., the outer surface of the tube is negatively charged and the inner surface of the hollow tube is positively charged. The halloysite is present in the form of nanotubes.

In one preferred embodiment of the invention, the halloysite is selected from halloysite-7 Å, halloysite-10 Å, and/or natural mixtures thereof. It is known to a person skilled in the art here that Å is the abbreviation for Angstrom and accordingly represents a physical length.

It would not be obvious to a person skilled in the art that the preferred nanotubes have a surprisingly good styptic effect, so that the paste also achieves an advantageous effect under wound healing aspects, in addition to the x-ray contrast capability. The halloysite nanotubes are furthermore biocompatible and are not subject to the EU nanoparticles definition (>50%; <100 nm) and are classified by the EPA as non-toxic/GRAS.

In a further preferred embodiment, the paste includes mineral materials comprising barite (BaSO₄) and halloy site in a ratio of:
preferably 2 to 6 volume parts barite to approximately one volume part halloysite; and
more preferably 3 to 5 volume parts barite to approximately one volume part halloysite; and
in particular approximately 4 volume parts barite to approximately one volume part halloysite.

The described volume ratios of barite and halloysite advantageously enable the visibility on an x-ray image and also a positive effect on the wound healing. It was surprising that the described volume components of the paste provide good temperature stability.

In one preferred embodiment, the paste preferably has a higher volume component of barite than halloysite. The ratio can thus also be designed as follows, for example (without being restricted thereto):
approximately 2 volume components barite to approximately one volume component halloysite; or
approximately 3 volume components barite to approximately 2 volume components halloysite; or
approximately 4 volume components barite to approximately 3 volume components halloysite; or
approximately 5 volume components barite to approximately 2 volume components halloysite.

The high volume component of barite ensures good visibility or contrast capability on an x-ray image, since in particular barite has good absorption properties. Moreover, barite is a frequently occurring mineral, so that it is advantageously acquired cost-effectively.

In one preferred embodiment of the invention, the paste is characterized in that the paste comprises a water component and a binder component. An improved consistency is advantageously displayed due to the addition of binder and water (viscosity at ambient pressure and ambient temperature: 5 mPa s to 2×10⁶ mPa s) for the further processing of the paste and advantageous binding of the individual paste components. The water component and the binder component can be added variably, so that the viscosity and also the binding of the paste components are changeable depending on requirements. A particularly smooth surface of a plastic to be marked requires, for example, a higher binder component in the paste than a porous surface structure of a textile to be marked in order to be able to produce optimum binding between the paste and the respective object to be marked.

According to the invention, a binder is a material which produces and/or promotes chemical bonds at phase boundaries of other materials and/or triggers and/or increases effects such as cohesion, absorption, and adhesion or friction. A binder binds materials in that it absorbs, accumulates, holds together, cross-links, or adhesively bonds them.

In one preferred embodiment, the water is demineralized water. Demineralized water has a very low electrical conductivity. This advantageously prevents the agglomeration/clumping of particles in the paste.

In a further preferred embodiment of the invention, the paste is characterized in that the paste comprises at least one color pigment. The advantage of such a paste is the visibility to the human eye outside an x-ray activity. Due to a strong contrast between the color of the paste and the textile fabric and/or a product on which the paste is applied, a user can clearly see which textile fabrics and/or products have been marked. A specific meaning can be assigned to each color. The paste can be made, for example red, blue, yellow, green, orange, turquoise, or brown. The color blue could indicate in wound dressings, for example, the use for the treatment of chronic wounds. Green-marked pieces of baggage could be classified in the airline industry, for example, as baggage already checked and classified as harmless.

In the meaning of the invention, color pigments are a coloring substance. In contrast to colorants, they consist of particles and are practically insoluble in the application medium. An application medium is the material into which the pigment is incorporated. According to the invention, the application medium is a paste of the type mentioned at the outset.

In one preferred embodiment, the paste does not comprise color pigments, so that it is not visible or is only recognizable with difficulty to the human eye after application on the textile fabric and/or product incapable of x-ray contrast. In contrast, the marking becomes visible on an x-ray image under the influence of x-rays. Therefore, the marking can advantageously be used as a so-called watermark. Watermarks could be used, for example, in order to make product piracy which is not recognizable upon first inspection visible under x-rays. A watermark could also be used to mark one's own products and recognize forgeries.

In a further preferred embodiment of the invention, the paste according to one of the preceding claims is characterized in that the primary particle size of barite and/or halloysite is <5 μm. The paste thus has good properties with respect with density and viscosity. In particular, small particle sizes have the advantage that they ensure good thorough mixing of the paste. Particles (solids) typically sink slowly to the bottom due to their greater density in comparison to the pure liquid and form a sediment (sedimentation). It was not predictable and therefore surprising to the inventors that at the described particle size of <5 μm, sediments can be essentially advantageously avoided.

In one preferred embodiment of the invention, it is provided that the halloysite nanotubes have the following dimensions: internal diameter: 10 to 20 nm, external diameter: 50 to 70 nm, and/or length: 0.3 to 4 μm. It has advantageously been shown that these dimensions occur in nature and therefore only have to be further processed for use in the paste using typical methods, such as drying, grinding, sifting, and dispersing.

According to the invention, primary particles are particles which can conglomerate to form larger composite systems (agglomerates or aggregates). A person skilled in the art knows that the particle sizes can vary and the primary particle size is preferably an average value. In one preferred design, the primary particle size of barite and halloysite is <10 μm, more preferably <8 μm, and in particular <5 μm. The advantage of the described particle sizes is the possibility of good and uniform thorough mixing of the particles in the suspension (paste).

In a further preferred embodiment of the invention, the paste is characterized in that the halloysite particles include a coating, which is applied by means of plasma technology and includes atomic copper. Advantageously, the applied copper surprisingly has an antibacterial effect. The antibacterial effect is advantageous for the treatment of chronic wounds.

It is known to a person skilled in the art that the coating of particles is also to be understood as a coating. Coating is to be understood according to the invention as the coating or encapsulation of particles with liquids (solution, suspension, or melt). In addition to the plasma technology, particles can also be coated by a fluidized bed coating method, a drum coating method, top spray coating methods, bottom spray coating methods, and a Wurster method. These methods are known to a person skilled in the art.

In one preferred embodiment, the vacuum-based plasma coating is applied to the halloysite particles using low-pressure plasmas for the application of a coating, wherein in particular PVD technology is used and sputter deposition in shaking chamber or rotating drum reactors is employed. The copper particle size on the halloysite surfaces is 5-10 nm.

It is known to a person skilled in the art that atomic copper is in particular a synonym for uncharged (charge-neutral) copper.

In a further preferred embodiment of the invention, the paste is characterized in that the binder includes a cellulose-saccharide mixture or is an acrylate. These binders prove to be particularly inexpensive and moreover have particularly good binding properties for the components of the paste. Acrylate has surprisingly rapid curing properties, so that the drying time can thus advantageously be kept short. The cellulose-saccharide mixture, in contrast, is advantageously very environmentally friendly.

Binders ensure the cohesion of powder particles. Binder solutions such as starch pastes and cellulose derivatives are most frequently used. Polysaccharide solutions having low viscosity are preferred due to the better disintegration-promoting property (rapid solution or disintegration in water). In the case of moisture-sensitive active ingredients, binders (cellulose derivatives, e.g., cellulose acetate, methyl cellulose, ethyl cellulose, hydroxypropyl methylcellulose, etc.) in organic solvents are used. Microcrystalline cellulose can also exert binder properties in dry form, whereby a water component is advantageously not necessary for binding halloysite and barite.

In one preferred embodiment of the invention, the mixture ratio of the cellulose-saccharide mixture is designed so that both cellulose and also saccharide are included by at least 0.01 wt.-% in relation to the total weight. The mixture preferably only comprises the two materials (cellulose and saccharide). The mixture advantageously has good biocompatibility, with surprisingly good binding.

In another preferred embodiment, it is provided that the binder comprises 90% to 95% deionized water and 5% to 10% acrylate. This advantageously permits the paste to bind particularly well to the textile fabric and/or product or to fix it there. It was surprising that no further additives were required for this purpose.

In a further preferred embodiment of the invention, the paste is characterized in that the paste comprises a water component of ≥65% by weight, a mineral material component of ≤30% by weight, a binder component of ≤8% by weight, and a color pigment component of ≤0.02% by weight. In particular these component quantities result in a paste which ensures the visibility under the influence of x-rays in a particularly inexpensive and high-quality manner, wherein advantageously little energy is required for producing and further processing the paste. The described quantity of color particles is surprisingly well sufficient to provide the paste with a uniform coloration.

In a further preferred embodiment, the paste comprises the following quantity components: a water component of approximately 55% by weight to approximately 65% by weight, a mineral material component of approximately 30% by weight to approximately 45% by weight, a binder component of approximately 8% by weight to approximately 10% by weight, and a color pigment component of approximately ≤0.02% by weight.

Furthermore, the invention includes a wound dressing, comprising composite fibers and a paste of the type mentioned at the outset applied thereon.

In the meaning of the invention, wound dressings are generally to be considered to be textile fabrics. The wound dressing according to the invention can preferably also be used as a bandaging means. In the meaning of the invention, this refers to those materials from which a medical bandage is produced. It is therefore also referred to as bandage material or colloquially as bandages. It is often stored easily accessible in the form of bandage boxes. Furthermore, it is possible to differentiate between sterile or at least sanitized wound dressings and nonsterile fastening material. Ready for use, combined compositions of wound dressing and fastening material are also preferred. Sterile wound dressings are compresses and bandage cloths. The fastening material preferably includes gauze bandages, Ideal-type bandages, elastic fixation bandages, knitted hosiery bandages, and triangular bandages.

Wounds and wound lesions are divided into various wound types and treated differently according to the effect and their appearance. For example, a cut arises due to a cut injury and is generally smooth-edged. Cuts usually bleed very strongly. They are among the most frequent injuries in everyday life. In addition to cuts, there are also lacerations, stab wounds, bites, gunshot wounds, contusions, abrasions, lacerations, scratches, burns, and chemical burns.

The advantage of a wound dressing which is marked using the above-described paste is the possibility of locating the wound dressing in x-ray images. The preferred wound dressing stops bleeding particularly efficiently in surgery, in first aid, in emergency care, or in the care of chronic wounds. It is not obvious to a person skilled in the art that the wound dressing according to the preferred embodiment results in improved care of an acute or chronic wound, since he could not presume that a small quantity of halloysite particles could already achieve such an effective action. It was surprising that the wound dressing according to the invention also minimizes wound hematoma and seroma. In addition, it was not predictable to the inventors that wound edges advantageously spread apart from one another less due to the use of the wound dressing of the mentioned type.

In one preferred embodiment, wound dressings are laid on external wounds in order to prevent the penetration of foreign bodies into the wound and absorb blood and wound exsudate. Moreover, wound dressings can ensure a moist and warm wound climate which promotes healing, reduce pain by way of contained substances, can promote wound healing, or can have an antimicrobial effect.

In a further embodiment, wound dressings are used for intracorporeal application. In particular in this case, the importance of visibility in x-ray images is highly relevant.

In a further preferred embodiment, the wound dressing is characterized in that the paste comprises 80% by volume barite and 20% by volume halloysite in relation to the total volume of the mineral materials.

In a further preferred embodiment, the paste includes
a) preferably 60-95% by volume barite and 5-40% by volume halloysite;
b) particularly preferably 70-85% by volume barite and 15-30% by volume halloysite; and
c) in particular 80% by volume barite and 20% by volume halloysite
   in relation to the total volume of the mineral materials.

Surprisingly, it has been shown precisely in the volume distribution of barite and halloysite that the marking of the wound dressing is very well visible in an x-ray image and nonetheless the advantageous properties of the halloysite are applied.

In another preferred embodiment, further volume component variants can also be comprised. It is always true according to the invention that barite has a greater volume component than halloysite in relation to a total volume of the mineral materials in the paste. Preferred components are described above. The large component of barite is advantageous for the visibility in an x-ray image, since the absorption rate of x-rays is advantageously high.

In a further preferred embodiment, the method for applying a paste to a textile fabric and/or other products is characterized in that the paste is applied by means of a printing method to the textile fabric and/or to the other product. Accurate, highly detailed patterns and a large quantity of textile fabrics and/or products can be printed at high speed by a printing method. Printing methods enable, in contrast in known spraying and immersion technologies of the prior art, exact and varying impregnation of the textile fabric surface, so that preferably the addition of further chemicals and agents, as is provided in the prior art, can be completely omitted. It was surprising that the applied particles of the paste advantageously do not dissolve out of the wound dressing.

In one preferred embodiment, the printing method is selected from the group of methods comprising: letterpress printing, gravure printing, screen printing, and/or flat printing. All methods are known to a person skilled in the art.

In one preferred embodiment, the method is characterized in that the printing method is a screen printing method and/or a rotation printing method. Both methods are known from the prior art. The paste of the type mentioned at the outset may be applied particularly well using such printing methods to a textile fabric and/or other product incapable of x-ray contrast. The advantage of the screen printing method is that the paste application can be varied by way of various levels of fabric fineness, so that high paste layer thicknesses can be achieved for the marking. Moreover, inflexible rigid objects can also be printed using the screen printing methods. Rotation printing methods advantageously enable particularly rapid printing of textile fabrics.

In one preferred embodiment, the method is characterized in that various printing motifs can be selected. Printing motifs can advantageously have a specific meaning, so that items of information about the printed textile fabric and/or product incapable of x-ray contrast can be provided to the user by a brief glance. Such items of information could, for example, warn of a hazard, have an address, or display the result of a check carried out at an earlier time, for example.

In one preferred embodiment of the invention, the printing motifs are designed as strips and/or waves. However, they can also comprise more detailed patterns or images by way of the above-mentioned printing methods. For example: a cross, a triangle, an image of the marked object, a pictogram, and much more.

In one preferred embodiment, the method is characterized in that after the printing of the paste, temperature-controlled drying is carried out. In the meaning of the invention, the temperature-controlled drying comprises monitored drying. This means the paste is dried after the printing at a defined temperature. The temperature is preferably uniformly at equal level everywhere in the drying chamber. This temperature is preferably the drying temperature. The advantage of such monitored drying is the process monitoring which is thus enabled. In addition, a regulated process is thus first possible, which is repeatable for each print. The process monitoring advantageously increases the quality of the products, so that the paste has an increased resistance (adhesion on the products to be printed).

In one preferred embodiment, the method is characterized in that the drying is executed at a drying temperature of <100° C. The comparatively low temperature (in comparison to the methods of the prior art described at the outset) for drying the applied paste advantageously requires little energy and is nonetheless sufficient for adhesion of the paste on the textile fabric and/or product.

In a further preferred embodiment of the invention, the drying temperature can be selected from the following temperature ranges: <98° C., <96° C., <95° C., or <90° C. Although low temperatures cause a longer drying time, the total energy consumption is surprisingly nonetheless lower. The described drying temperatures advantageously enable uniform drying of the paste, whereby the resistance increases.

In a last aspect, the invention includes a use of the above-mentioned wound dressing for the treatment of wound lesions.

In one preferred embodiment of the use of the wound dressing according to the invention, strongly bleeding wounds are first cleaned, subsequently a pressure bandage is preferably applied over the sterile wound dressing according to the invention to avoid a greater blood loss.

1. A paste for marking textile fabrics and/or other products incapable of x-ray contrast, characterized in that the paste includes mineral materials, comprising barite (BaSO4) and halloysite in a ratio of four volume parts barite to one volume part halloysite.

2. The paste as claimed in claim 1, characterized in that the paste comprises a water component and a binder component.

3. The paste as claimed in claim 1, wherein the paste comprises at least one color pigment.

4. The paste as claimed in claim 1, wherein the primary particle size of barite and/or halloysite is <5 μm.

5. The paste as claimed in claim 1, wherein the halloysite particles include a coating, which is applied by means of plasma technology and includes atomic copper.

6. The paste as claimed in claim 1, wherein the binder includes a cellulose-saccharide mixture or is an acrylate.

7. The paste as claimed in claim 1, wherein the paste comprises a water component of greater than or equal to 65% by weight, a mineral material component of less than or equal to 30% by weight, a binder component of less than or equal to 8% by weight, and a color pigment component of less than or equal to 0.02% by weight.

8. A wound dressing comprising composite fibers and a paste applied thereon according to claim 7.

9. The wound dressing as claimed in claim 8, characterized in that the paste comprises 80% by volume barite and 20% by volume halloysite in relation to the total volume of the mineral material component.

10. A method for applying a paste as claimed in claim 1 to a textile fabric and/or other products, characterized in that the paste is applied by means of a printing method to the textile fabric and/or to the other product.

11. The method as claimed in claim 10, characterized in that the printing method is a screen printing method and/or a rotation printing method.

12. The method as claimed in claim 10, characterized in that various printing motifs can be selected.

13. The method according to claim 10, wherein after the printing of the paste, a temperature-controlled drying is carried out.

14. The method according to claim 10, wherein the drying is executed at a drying temperature of <100° C.

15. A use of the wound dressing as claimed in claim 8 for the treatment of wound lesions.
